(19) United States
(12) Patent Application Publication
(10) Pub. No.: US 2005/0070873 A1
(43) Pub. Date: Mar. 31, 2005

(54) Constituent delivery system

(75) Inventor: Jimmie L. Johnson, Chicago, IL (US)

Correspondence Address: Gerald White;Gerald K White and Assocs PC, Ste 835, 205 W Rondolph St, Chicago, IL, 60606, US

(73) Assignee: The Compak Companies, LLC

(21) Appl. No.: 10/990,872

(22) Filed: Nov. 17, 2004

Related U.S. Application Data
(63) Continuation of application No. 09/633,688, filed on Aug. 7, 2000.
Application No. 09/633,688 is a continuation-in-part of application No. 08/979,028, filed on Nov. 26, 1997, now abandoned.
Application No. 08/979,028 is a continuation of application No. 08/641,430, filed on May 1, 1996, now abandoned.
(60) Provisional application No. 60/004,967, filed on Oct. 10, 1995.

(51) Int. Cl. A61M 5/00

(52) U.S. Cl. 604/415

(57) ABSTRACT

A constituent delivery vial and method employs a sealed vial portion which employs an inner lid to keep a first constituent, suitably a liquid, separate from a second constituent. The second constituent is contained within a capsule wherein the capsule is maintained within a chamber between the first lid and an outer top lid. The constituents are mixed by depressing the top lid to cause the capsule to penetrate the bottom lid and either directly release the constituent containers in the capsule to the constituent contained below the lower lid or, in the case of a dissolving capsule, to allow combining of the two once the capsule dissolves. The bottom of the cup includes a syringe well which slopes downwardly to provide a central lowest point to the cup to enable use of a syringe for retrieval of as much of the mixed constituents as possible. Another embodiment of the constituent delivery vial and method includes use of a third lid that is in sealed relationship and covers the outer top lid. The third lid is at least partially removable and serves to provide further sterilization.

This patent application is a continuation of patent application Ser. No. 09/633,688, filed Aug. 7, 2000, which is a continuation-in-part of patent application Ser. No. 08/979,028, filed Nov. 26, 1997, which is a file wrapper continuation of patent application Ser. No. 08/641,430, filed May 1, 1996, which is a non-provisional of U.S. provisional patent application Ser. No. 60/004,967, filed Oct. 10, 1995.

### BACKGROUND OF THE INVENTION

In delivery of items, for example medicine or pharmaceuticals or food stuffs, especially when the items are shipped long distances, in order to ensure a long shelf life of the items, portions are often separated into constituent parts and then recombined just prior to use, for example with medicines such as antibiotics , portions are freeze dried and shipped separately from a sterile liquid component whereupon the liquid portion of the container is sterilized and the dried ingredients are added thereto and mixed and the liquid thus produced is then available for use with a syringe, for injecting into a patient for example, or for consuming orally if that is the appropriate mode of consumption.

An alternative method of employing such separate constituents which are later mixed is to have both the freeze dried component and the liquid component in separate sterile containers (for example, the liquid component may be in a prefilled syringe and the freeze dried component in a vial which includes a syringe-penetrable top). The vial top is sterilized through use of an alcohol swab, for example, and the syringe is caused to penetrate the top thereof so as to enable injection of the liquid into the vial. The syringe is then suitable withdrawn and the liquid and freeze dried component are mixed to create a well dispersed suspension or until the freeze dried component dissolves whereupon the vial top may again be sterilized through use of an alcohol swab and the liquid pharmaceutical then be withdrawn in the appropriate amount with the syringe, for injection into the patient.

Such systems are at times less than desirable, since the separate shipping or packaging of the various components is prone to situations where one constituent is shipped and separated from the other constituent, resulting in difficulties in keeping the two constituents together. Further, the chance of contamination or infection of the ingredients is greater, since the number of steps where sterility must be maintained is increased, for example, the syringe must be maintained sterile, the contents of the syringe must be maintained in a sterile condition, the freeze dried component must be maintained in a sterile condition and the external surfaces of the containers which may contact various parts of the syringe or the like must also be maintained or made sterile, increasing the chance of contamination. Another drawback arises when the liquid portion is typically provided in bulk and must be measured at the time of reconstituting with sterile measuring. Such remixing may be difficult in filed locations which may not have access to sterile conditions.

### SUMMARY OF THE INVENTION

According to the present invention, a system of at least two constituents is provided in a unitary container wherein a liquid constituent, for example, is contained within a vial which may take the shape of a cup and an inner lid portion seals the liquid constituent within its container. An outer lid is also provided with a space defined between the inner and outer lid wherein the other constituent or constituents are contained within the space therebetween. In use, the other constituent is caused to penetrate the inner lid so as to combine with the liquid ingredient, while the outer lid is maintained in its sealed state. The constituents are still maintained sterile within the vial or cup. Mixing then occurs whereupon the outer lid may be removed, or alternatively, may be sterilized with an alcohol wipe and a syringe inserted therein.

It is an object of the invention to provide an improved delivery system and method for providing multipart constituents.

It is a further object of the present invention to provide an improved system for providing pharmaceuticals with separated portions while enabling reconstituting the portions in a sterile environment.

It is an object of the present invention to provide an improved method and system for enabling premixing of constituents in premeasured portions prior to use.

### BRIEF DESCRIPTION OF THE DRAWINGS

FIG. 1 is a cross sectional view of a container according to the present invention for providing two constituents;

FIG. 2 is a cross sectional view of the container of FIG. 1 after the ingredients are combined;

FIG. 3 is a view of the container of FIG. 2 illustrating removal of the now mixed components via use of a syringe;

FIG. 4 is a view of the capsule portion of the container of FIG. 1;

FIG. 5 is a view of an alternative capsule portion;

FIG. 6 is a view of a preferred embodiment container employing an additional lid for sanitary purposes; and

FIGS. 7-9 are views illustrating the use of the embodiment of FIG. 6.

### DETAILED DESCRIPTION

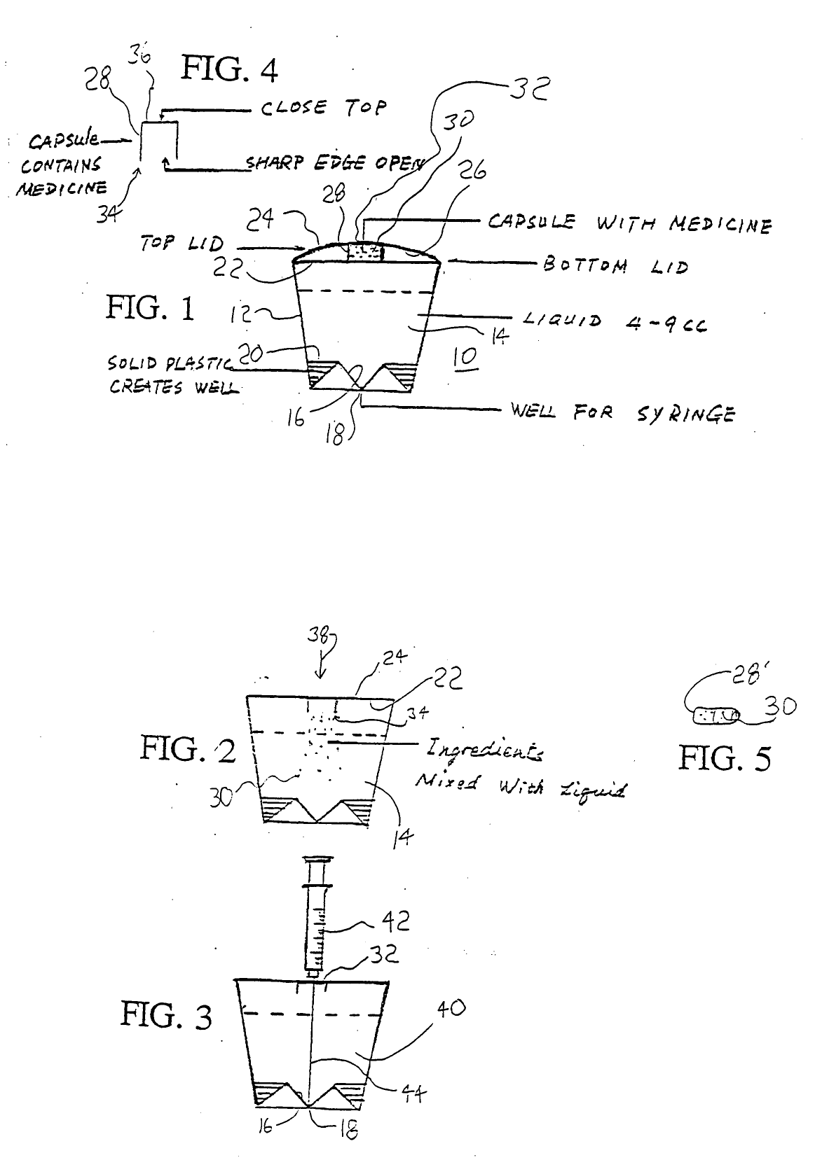

Referring now to FIG. 1, a container 10 in accordance with the present invention comprises a cup-like container 12, which suitably is made of plastic wherein a liquid 14 is contained within the cup. In the illustrated embodiment, the liquid comprises 4-9 ccs in volume. The interior bottom of the cup defines a well 16 therein with inwardly sloping walls so as to ensure that the center 18 of the well is the lowest most point within the interior of the cup or vial 12. The interior of the cup may comprise solid portions 20 which assist in defining the shape of the well.

Near the top of the cup is provided an inner lid 22 which substantially seals the interior of the liquid containing chamber and an outer lid 24 which is also secured to the cup 12 or to the top of bottom lid 22. A central cavity 26 is suitably defined between the top lid 24 and the lower lid 22 wherein a capsule member 28 is positioned within the chamber. The capsule may contain a medicine or freeze dried constituent of an antibiotic, for example, 30 therewithin. In this state, the two constituents 14 and 30 are maintained separate from each other, but are sealed to the outside world so as to ensure sterility. The region of top lid 24 above capsule 28 may suitable comprise a rubber stopper portion 32o the type employed in vials wherein a syringe may be inserted therein as necessary.

Referring now to FIG. 4, the structure of the capsule portion 28 may be observed. The capsule may suitable be of plastic or glass or any other suitable material and includes sharp edge portions 34 and sealed top portion 36, which may also include a rubberized stopper of the type normally employed in conjunction with vials adapted for use with syringes. The constituent is suitable confined with the capsule, but the bottom of the capsule remains opened. The bottom lid is thus relied upon to keep the medicine with the capsule, since the bottom lid would thus provide the bottom sealing portion to capsule 28.

Referring now to FIG. 2, in use, in order to mix the two constituents, top lid 24 is pushed downwardly in the direction of arrow 38 of FIG. 2 whereupon the sharp edge portions 34 of the capsule puncture the bottom lid. Since the bottom lid is suitable designed to be so punctured, the constituent 30 is released into the liquid 14 whereupon the two are mixed through vigorous shaking if appropriate. Note that the top lid seal has not been broken, so the interior of the cup 12 remains sterile or otherwise unopened to the outside atmosphere.

Referring now to FIG. 3, to retrieve the now mixed ingredients 40, which is the combination of the constituent 30 and 14, a syringe 42 is inserted through region 32 which suitable comprises a rubber stopper or the like as typically employed with syringe receiving vials (suitably after the top thereof is cleansed with an alcohol swipe or the like). The needle 44 of the syringe suitably extends down to the bottom 18 of well 16 so as to enable withdrawal of the maximum amount of the liquid constituent 40 as possible without excessive waste.

It should be noted that the container and its ingredients may be so measured as to either provide single dosage or multiple dose as desired. As an example, ingredients 14 and 30 may comprise a liquid constituent and a freeze dried antibiotic, vitamin components, or other pharmaceuticals. Also, the items together may comprise certain foodstuffs-or the like.

Referring to FIG. 5, an alternative embodiment employs a capsule 28′ which is suitably made of an inert ingredient that dissolves in the liquid, such as a gelatin type capsule typically used in the delivery of pharmaceuticals or vitamins. In this embodiment, the ingredient 30 is contained within the capsule and bottom lid portion 22 is sufficiently thin so as to enable the capsule 28′ to penetrate through the bottom lid when the top lid is pressed downwardly in the direction of arrow 38 (FIG. 2). The capsule 28′ then falls into the liquid portion 14 and suitably dissolves to release ingredient 30 to the liquid for subsequent mixing.

The lid portion 24 is also provided in an alternative embodiment in a deformable plastic which once pressed downwardly retains its deformed shape or becomes discolored so as to enable a determination to be made whether the capsule portion has been depressed into the liquid portion. Thus, on quick inspection, it is possible to determine whether the cup is a fresh unmixed vial or whether it has been lo previously mixed either intentionally or through damage during shipping.

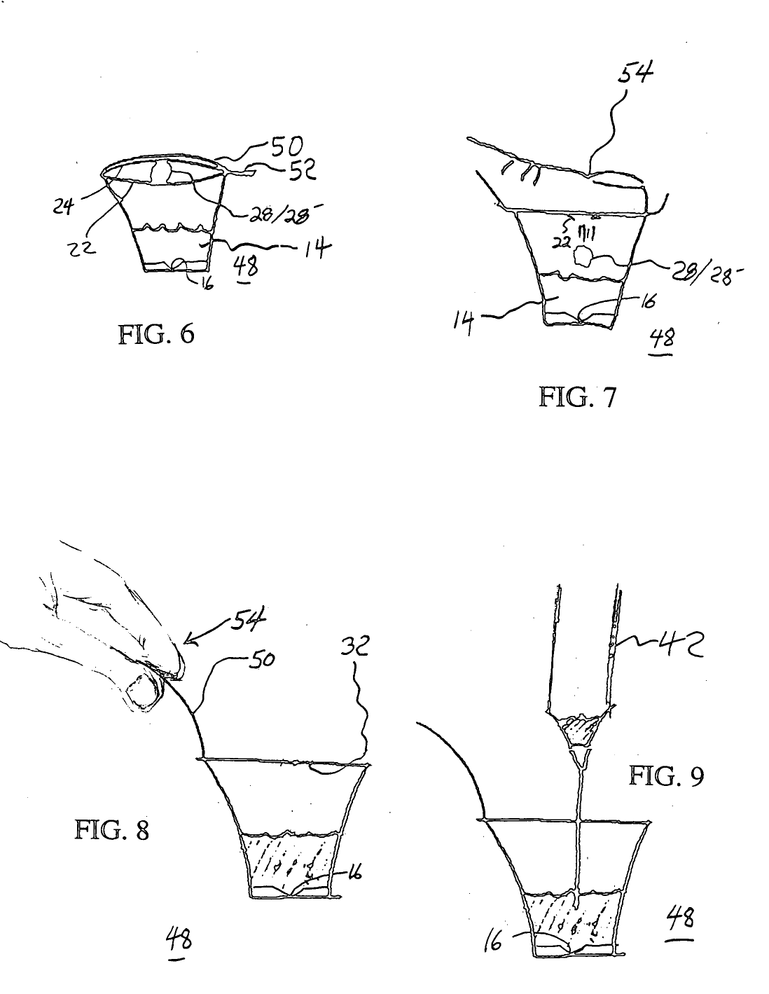

Referring now to FIG. 6, in a preferred embodiment of a container 48 according to the invention, another lid layer 50 is suitably provided as an outermost lid, on top of outer lid 24. Lid layer 50 is suitably peelable or otherwise removable, by lifting a tab member portion 52 thereof in an upwardly direction, to peel the lid away from covering the top layer of lid 24 and stopper portion 32. Lid layer 50 is advantageous in providing an additional sanitary feature, as may be understood in connection with FIGS. 7-9, which are views illustrating the embodiment of FIG. 6 in use. To employ the container 48 of the present invention, the user presses downwardly on the top of lid layer 50, as illustrated in FIG. 7, wherein the user's thumb or finger 54 is shown. The downward pressure causes capsule 28/28′ to penetrate lower lid 22, so as to be combined with the constituent 14 within the interior of the container. Next, as show in FIG. 8, the user peels lid layer 50 away from the top of the cup (either completely removing the layer 50, or leaving it partially attached as shown in FIG. 8). Since the top lid layer 50 was in place when the user pressed on the lid structure to release the capsule, the next layer down, which has the syringe receiving portion 32, is maintained clean and sterile, since it was not touched by the user's finger or thumb (layer 50 provided a sanitary shield between the user and the syringe receiving portion. Now, as shown in FIG. 9, the syringe 42 can be inserted into the interior of the container to draw the contents up into the syringe. Depending on the particular constituents in the container and capsule, it may be desirable to mix the compounds by shaking or the like, prior to withdrawing into the syringe.

While a single capsule 28/28′ is shown in the illustrated embodiments, plural such capsule may be employed, when more than two constituents are desirably separated until time of use.

Accordingly, the present invention provides an improved delivery system and method for providing separate constituents, suitably food or pharmaceutical ingredients, enabling longer shelf life while maintaining sterile conditions and enabling sterile mixing in the field. Also, with the container and system of the invention, an easy to use sanitary injectable constituent container is provided that maintains plural constituents separate until use, while enabling sanitary deployment, mixing and withdrawal of the mixed constituents via a syringe or the like.

1. A constituent delivery system for maintaining a liquid constituent and a solid constituent separate from each other until combining, said constituents comprising:
(a) a cup-like container having a closed bottom, side walls, and an open top and adapted to contain said liquid constituent;
(b) a first lid in sealed relationship with said open container top for separating said liquid and solid constituents, maintaining said liquid constituent sterile, and said first lid adapted to be broken under pressure applied through a third lid, a second lid, and said solid constituent;
(c) said second lid in sealed relationship with said first lid and having a central cavity adapted to contain said solid constituent between said first and second lids and maintaining said solid constituent sterile, said second lid having a syringe receiving stopper; and
(d) said third lid in sealed relationship with and covering said second lid to maintain said second lid sterile, said third lid being at least partially removable from said second lid.

2. The constituent delivery system of claim 1, wherein said solid constituent comprises a capsule.

3. The constituent delivery system of claim 1, wherein said container further comprises inwardly sloping walls to form a well at said closed bottom.

4. The constituent delivery system of claim 1, wherein said second lid is deformable.

5. The constituent delivery system of claim 1, wherein said third lid includes a tab for gripping to facilitate removal of said third lid by peeling from said second lid.

6. A method for combining a liquid constituent and a solid constituent while maintaining a sterile environment comprising:
(a) providing a constituent delivery system containing a liquid and solid constituent comprising:
i. a container having a closed bottom, sidewalls, and an open top and containing said liquid constituent;
ii. a first lid in sealed relationship with said open container top for separating said liquid and solid constituents and maintaining said liquid constituent sterile and adapted to be broken under pressure applied through a third lid, a second lid, and said solid constituent;
iii. said second lid in sealed relationship with said first lid and having a central cavity which contains said solid constituent between said first and second lids and maintaining said solid constituent sterile, said second lid having a syringe-receiving stopper; and
iv. said third lid in sealed relationship with and covering said second lid to maintain said second lid sterile and at least partially removable from said second lid;
(b) applying pressure to a portion of said third and said second lids so as to apply pressure to said solid constituent and thereby cause said solid constituent to penetrate said first lid and enter into said container where said liquid and solid constituents become combined and maintained in a sterile environment;
(c) removing at least a portion of said third lid to provide access to said second lid and said syringe-receiving stopper; and
(d) inserting a syringe into said container through said syringe-receiving stopper and withdrawing said combined constituents.

7. The method of claim 6, wherein said solid constituent comprises a capsule.

8. The method of claim 6, wherein said third lid is removed by peeling said third lid from said second lid.

9. The method of claim 6, wherein said combined constituents comprise a medicinal preparation.
